(19) United States
(12) Patent Application Publication
(10) Pub. No.: US 2006/0051393 A1
(43) Pub. Date: Mar. 9, 2006

(54) Method of manufacturing drug-eluting medical device

(75) Inventors: Kenneth T. Heruth, Edina, MN (US); Edouard Koullick, Golden Valley, MN (US); Mark S. Lent, Brooklyn Park, MN (US)

Correspondence Address: MEDTRONIC, INC., 710 MEDTRONIC PARK, MINNEAPOLIS, MN, 55432-9924, US

(73) Assignee: Medtronic, Inc., Minneapolis, MN (US)

(21) Appl. No.: 10/936,263

(22) Filed: Sep. 8, 2004

(51) Int. Cl. A61L 33/00; A61F 2/00

(52) U.S. Cl. 424/423; 427/2.24

(57) ABSTRACT

Methods for manufacturing medical devices comprising a polymeric material capable of releasing a therapeutic agent upon contact with bodily tissue or fluid are described. The method includes generating polymeric material having a matrix into or onto which a therapeutic agent is disposed and having pores. The method further includes disposing additional therapeutic agent into the pores of the polymeric material.

### FIELD

The present disclosure relates to porous polymer coatings of medical devices as vehicles for drug delivery.

### BACKGROUND

Implantation of medical devices, such as pacemakers, neurostimulators, implanted drug pumps, leads, catheters, etc, has been associated with adverse consequences, such as formation of scar tissue surrounding the implant, infection due to bacteria introduced during implantation, and tissue proliferation in blood vessels after a stent implantation. Attempts to prevent or control such adverse reactions have included administration of drugs, completely separate from the intended primary therapy of the implanted medical device. In some cases, systemically administered drugs, e.g. orally, intravenously, or intramuscularly administered drugs, have proven effective in treating complications due to medical device implantation. In other cases, systemic delivery has been ineffective due to, e.g., pharmacokinetic or pharmacodynamic characteristics of the drug, the location of the implanted device, or side effects of the drug. To increase effectiveness in these situations, some implanted devices have been modified to elute the drug into the surrounding tissues.

One common way of providing local drug elution is to dispose a polymer layer on the implantable medical device and embed the drug into the polymer during manufacturing. When hydrated after implant, the drug diffuses out of the polymer into surrounding tissue. Various methods of impregnating polymers with drugs have been used, including mixing the drug into the melted polymer prior to processing (e.g. molding or extrusion), and diffusing the drug into a finished polymer component using chemicals to swell the polymer for rapid loading. In some cases, the implantable medical device (IMD) is made from a polymer that is compatible with the drug, and the drug can be loaded directly into the device. However, many IMDs are made from metals or from polymers that are inherently incompatible with the desired drug. In such situations, the IMD can be coated with a thin layer of a compatible polymer, and the drug can be loaded into the coating layer.

However, problems exist with current loading technology. For example, it can difficult to load large quantities of drugs or to adjust release rates when conventional biomaterials (silicone rubber, polyurethane, etc) are used as a matrix for drug loading.

### BRIEF SUMMARY

An embodiment of the invention provides a method for manufacturing a medical device. The method comprises disposing about, on, and/or in at least a portion of an external surface of a medical device a polymeric material comprising a beneficial agent and pores. The method further comprises disposing on and/or in the pores additional beneficial agent.

An embodiment of the invention provides a method of manufacturing a medical device. The method comprises generating a polymeric material comprising a beneficial agent and pores; disposing the polymeric material on, in, and/or about at least a portion of an external surface of the medical device; and disposing additional therapeutic agent on and/or in the pores.

An embodiment of the invention provides a method of manufacturing a medical device. The method comprises disposing a polymeric material comprising a therapeutic agent and a porogen about, on, and/or in at least a portion of an external surface of the medical device; removing the porogen from the polymeric material to produce a polymeric layer comprising pores; and disposing on and/or in the pores additional therapeutic agent.

An embodiment of the invention provides a method of manufacturing a medical device. The method comprises disposing a polymeric material comprising a therapeutic agent about, on, and/or in at least a portion of an external surface of the medical device; creating pores in the polymeric material; and disposing on and/or in the pores additional therapeutic agent.

An embodiment of the invention provides a method of manufacturing a medical device. The method comprises generating a polymeric material comprising a therapeutic agent and pores, and disposing additional therapeutic agent on and/or in the pores to produce a loaded polymeric material comprising loaded pores. The method further comprises disposing the loaded polymeric material comprising loaded pores on or about at least a portion of an external surface of the medical device.

Advantages of at least some embodiments of the invention may include the ability to modify the release profile of one or more beneficial agents and/or the ability to enhance the quantity of one or more beneficial agents to be released. For example, by loading a polymeric material with a beneficial agent prior to disposing the polymeric material on, in, and/or about at least a portion of a medical device and then loading additional beneficial agent into and/or on pores of the pre-loaded polymeric material, enhanced loading of the beneficial agent may be accomplished. In addition, preloading a polymeric material with a beneficial agent and loading beneficial agent into and/or on pores of a the polymeric material allows for better control over the release rate of beneficial agent, both long term (within the polymeric matrix) and short term (in pores). These and other advantages will become evident to one of skill in the art upon reading the disclosure herein.

### BRIEF DESCRIPTION OF THE DRAWINGS

FIG. 1 is a diagrammatic illustration of a cross-section of polymeric material comprising porogen.

FIG. 2 is a diagrammatic illustration of a cross-section of polymeric material comprising pores.

FIG. 3 is a diagrammatic illustration of a cross-section of polymeric material comprising therapeutic agent and porogen.

FIG. 4 is a diagrammatic illustration of a cross-section of polymeric material comprising therapeutic agent and pores.

FIG. 5 is a diagrammatic illustration of a cross section of a portion of a device or polymeric material comprising therapeutic agent, pores, and additional therapeutic agent in the pores.

FIG. 6A is a diagrammatic illustration of a cross section of a portion of a device comprising a surface layer and polymeric material comprising pores, the polymeric material being disposed on or about the surface layer.

FIG. 6B is a diagrammatic illustration of a cross section of a portion of a device comprising a surface layer and polymeric material comprising therapeutic agent and pores, the polymeric material being disposed on or about the surface layer.

FIG. 6C is a diagrammatic illustration of a cross section of a portion of a device comprising a surface layer and polymeric material comprising therapeutic agent, pores, and therapeutic agent in the pores, the polymeric material being disposed on or about the surface layer.

FIG. 7A is a diagrammatic illustration of a cross-section of a portion of a device comprising a surface layer, and intermediate layer disposed on or about the surface layer, and a polymeric material comprising pores disposed on or about the intermediate layer.

FIG. 7B is a diagrammatic illustration of a cross-section of a portion of a device comprising a surface layer, and intermediate layer disposed on or about the surface layer, and a polymeric material comprising therapeutic agent and pores disposed on or about the intermediate layer.

FIG. 7C is a diagrammatic illustration of a cross-section of a portion of a device comprising a surface layer, and intermediate layer disposed on or about the surface layer, and a polymeric material disposed on or about the intermediate layer, the polymeric material comprising therapeutic agent, pores, and additional therapeutic agent in the pores.

FIG. 8A is a diagrammatic illustration of a cross-section of a portion of a device comprising a surface layer comprising therapeutic agent and polymeric material disposed on or about the surface layer, the polymeric material comprising therapeutic agent, pores, and additional therapeutic agent in the pores.

FIG. 8B is a diagrammatic illustration of a cross-section of a portion of a device comprising a surface layer comprising therapeutic agent, an intermediate layer disposed on or about the surface layer, and polymeric material disposed on or about the intermediate layer, the polymeric material comprising therapeutic agent, pores, and additional therapeutic agent in the pores.

FIG. 8C is a diagrammatic illustration of a cross-section of a portion of a device comprising a surface layer comprising therapeutic agent, an intermediate layer comprising therapeutic agent disposed on or about the surface layer, and polymeric material disposed on or about the intermediate layer, the polymeric material comprising therapeutic agent, pores, and additional therapeutic agent in the pores.

FIG. 8D is a diagrammatic illustration of a cross-section of a portion of a device comprising a surface layer, an intermediate layer comprising therapeutic agent disposed on or about the surface layer, and polymeric material disposed on or about the intermediate layer, the polymeric material comprising therapeutic agent, pores, and additional therapeutic agent in the pores.

FIG. 9-13 are flow diagrams according to embodiments of the invention.

The drawings are not necessarily to scale. Like numbers refer to like parts or steps throughout the drawings.

### DETAILED DESCRIPTION

In the following description, reference is made to the accompanying drawings that form a part hereof, and in which are shown by way of illustration several specific embodiments of the invention. It is to be understood that other embodiments of the present invention are contemplated and may be made without departing from the scope or spirit of the present invention. The following detailed description, therefore, is not to be taken in a limiting sense.

Various embodiments of the present invention relate to manufacture of implantable medical devices capable of eluting a therapeutic agent from a surface of the device when implanted in a patient. In some embodiments, the device comprises a polymeric material disposed about or on at least a portion of the device. The polymeric material comprises a polymeric matrix substrate and pores. Therapeutic agent is disposed on or in the matrix, which allows for longer-term release of the therapeutic agent. Therapeutic agent is also disposed in the pores, allowing for more rapid release of therapeutic agent after implantation. In various embodiments, methods allow for individual control of the loading of therapeutic agent into the matrix and loading of the pores. Accordingly, the amount of therapeutic agent loaded into or on the polymeric material and the release profile of therapeutic agent from the implantable device can be more closely controlled.

It should be understood that, as used herein “implanted medical device”, “implantable medical device”, and the like refer to medical devices that are to be at least partially placed within a patient's body. Typically, such devices, or portions thereof, are placed within the patient's body for a period of time for which it would be beneficial to have a therapeutic agent present on a surface of the device. For example, a medical device implanted in a patient's body for several hours or more constitutes an implantable medical device for the purposes of this disclosure.

Overview

Any implantable medical device or system may be manufactured according to the teachings of the present disclosure. Non-limiting examples of implantable medical devices include leads, catheters, lead extensions, infusion pumps, pulse generators, defibrillators, pacemakers, stents, bone grafts, and the like.

In general, medical devices made in accordance to the present disclosure comprise a polymeric material comprising a polymeric matrix and pores. Therapeutic agent is disposed in or on the polymeric matrix and in the pores. The polymeric material may be disposed on or about at least a portion of a surface of an implantable medical device. Alternatively, the surface of the device may comprise the polymeric material. Various exemplary ways of manufacturing such devices are disclosed herein.

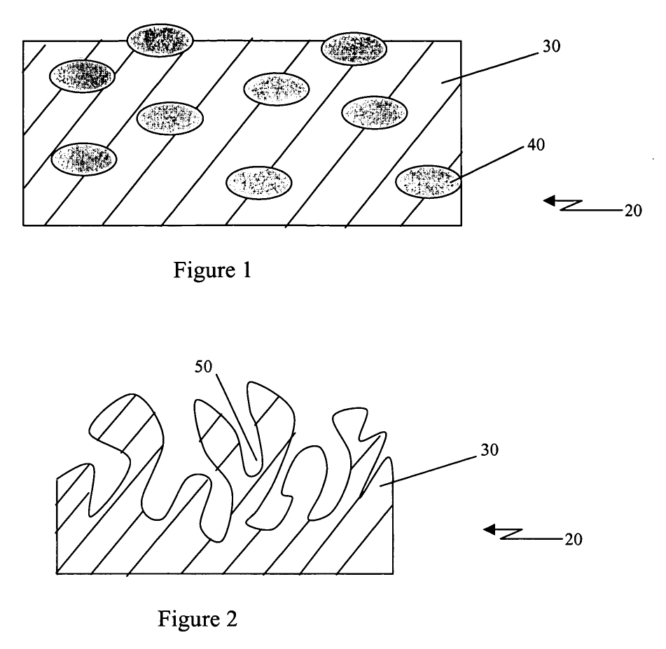

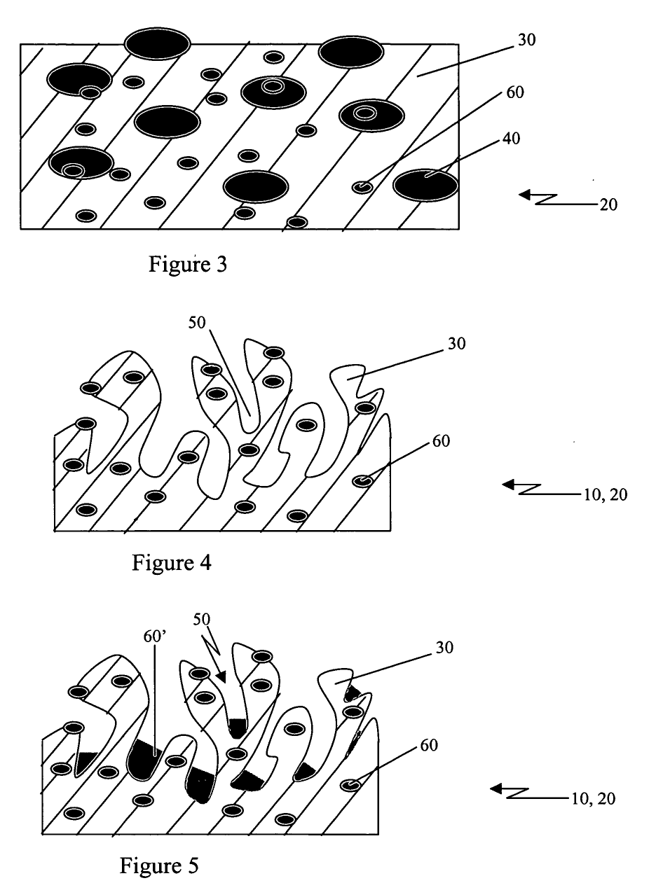

Referring to FIG. 1, a cross-section of polymeric material 20 comprising a polymeric matrix and porogen 40 is shown. The porogen 40 may be removed yielding a polymeric material 20 comprising pores 50 (FIG. 2). Alternatively, the pores 50 may be created by, e.g., foaming, mixing with gas, or curing or setting in high humidity, which techniques are discussed in more detail below.

Referring to FIG. 3, a cross-section of a polymeric material 20 comprising a polymeric matrix 30, porogen 40, and therapeutic agent 60 disposed in or on the matrix 30. The porogen 40 may be removed yielding a polymeric material 20 comprising therapeutic agent 60 and pores 50 (FIG. 4). Alternatively, the pores 50 may be created by, e.g., foaming, mixing with gas, or curing or setting in high humidity, which techniques are discussed in more detail below.

Referring to FIG. 4, polymeric material 20 comprising a polymer matrix 30, pores 50, and therapeutic agent 60 in or on the matrix 30 is shown. Polymeric material 20 as shown in FIG. 4, may be prepared using any known or future developed technique or process. For example, therapeutic agent 60 may be mixed with polymeric matrix 30 material prior to curing or setting. A porogen may also be mixed with the polymeric matrix 30 material and therapeutic agent. The porogen may then be removed to yield a polymeric material 20 as show in FIG. 4. The polymeric material 20 may alternatively be made porous by, e.g., extruding in the presence of gas, such as CO₂; setting or curing in high humidity; foaming prior to extrusion; or the like. Alternatively, polymeric matrix 30 material may be made porous prior to introduction of therapeutic agent 60. Therapeutic agent 60 may then be introduced into or on polymer matrix 30 by, e.g., a solvent-swelling technique.

Referring to FIG. 5, polymeric material 20 comprising a polymer matrix 30, therapeutic agent 60, and additional therapeutic agent 60′ in pores 50 is shown. In various embodiments, at least a portion of a surface layer of a device 10 may comprise polymeric material 20 as shown in FIG. 5. Non-limiting examples of such devices 10 include catheters and leads having bodies made of polymeric material 20. Polymeric material 20 and devices 10, or portions thereof, as shown in FIG. 5, may be prepared using any known or future developed technique or process. For example, a polymeric material 20 comprising a polymeric matrix 30, pores 50, and therapeutic agent 50 may be made as discussed above with regard to FIG. 4. Additional therapeutic agent 60′ may then be introduced into pores 50. One way of introducing additional therapeutic agent 60′ into pores 50 includes mixing additional therapeutic agent 60′ in a solvent and contacting the polymeric material 20 comprising pores 50 with the mixed solvent and additional therapeutic agent 60′. The solvent may be dried leaving additional therapeutic agent 60′ in pores 50. The solvent may or may not be a solvent that allows penetration of additional therapeutic agent 60′ into polymeric matrix 30.

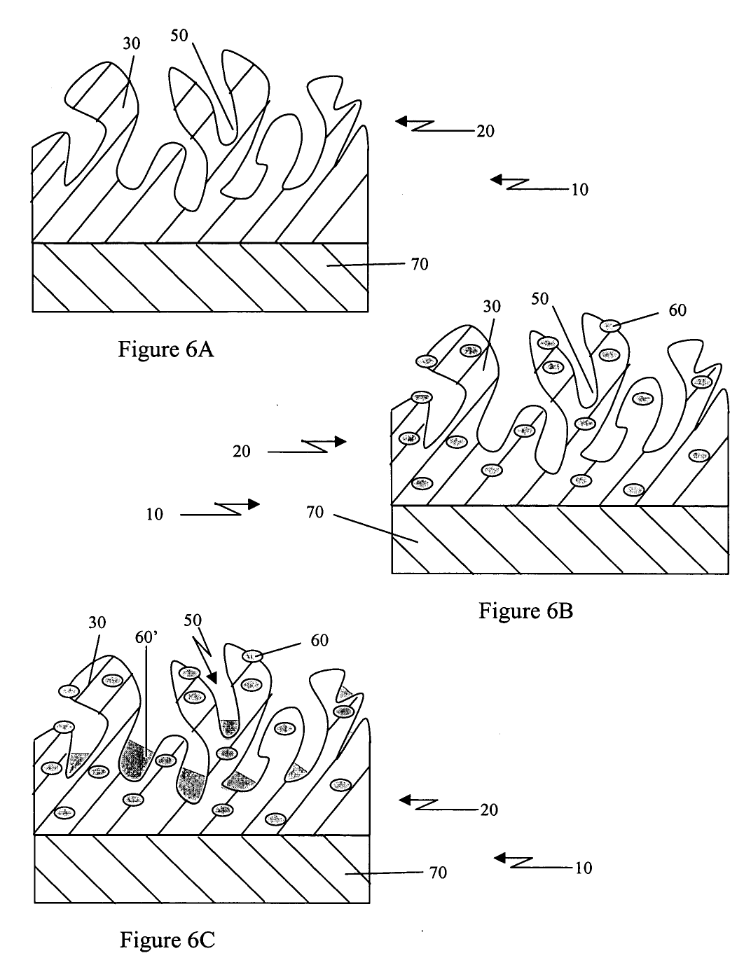

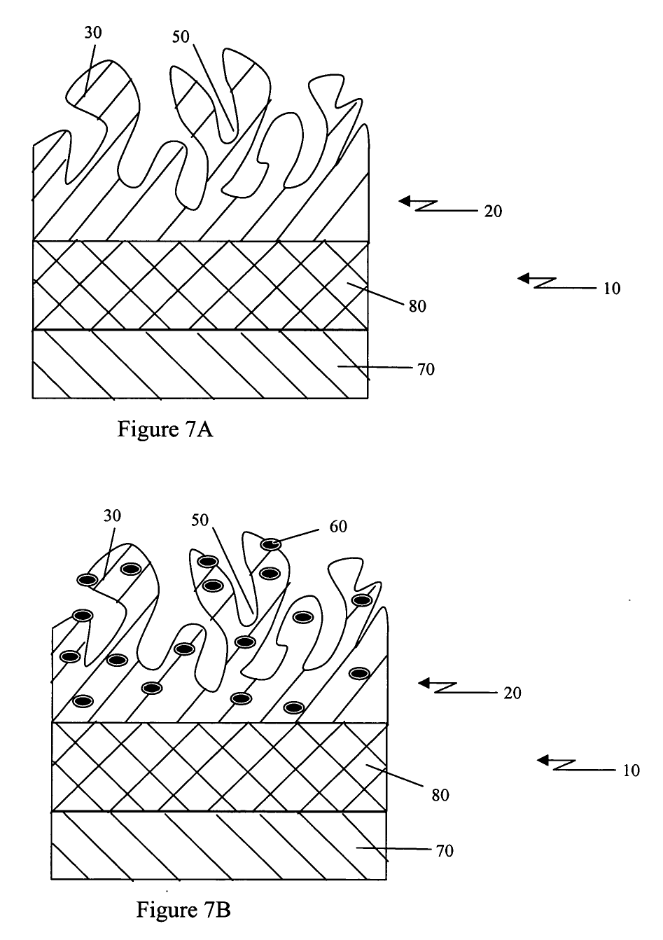

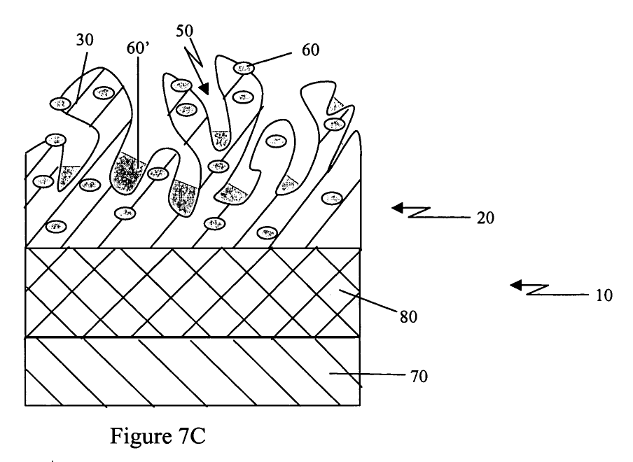

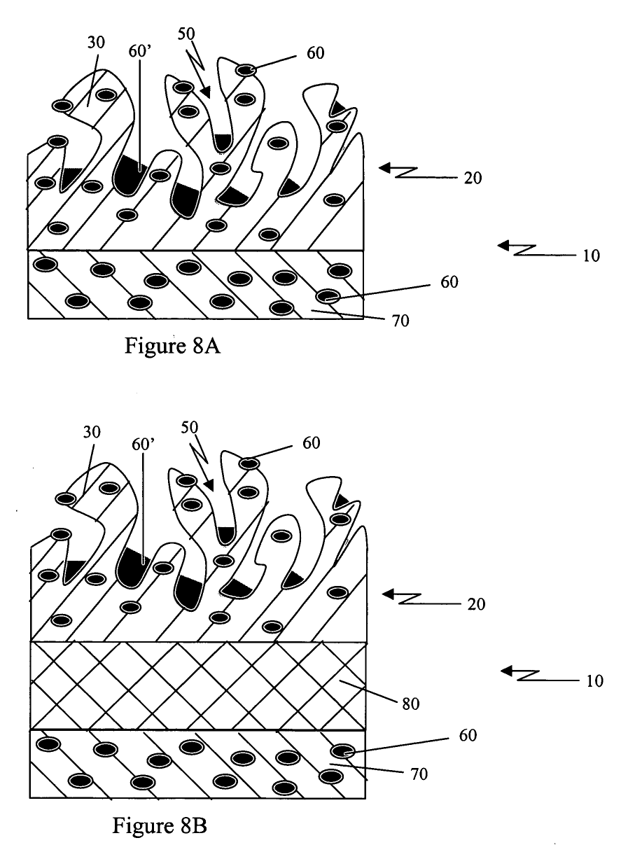

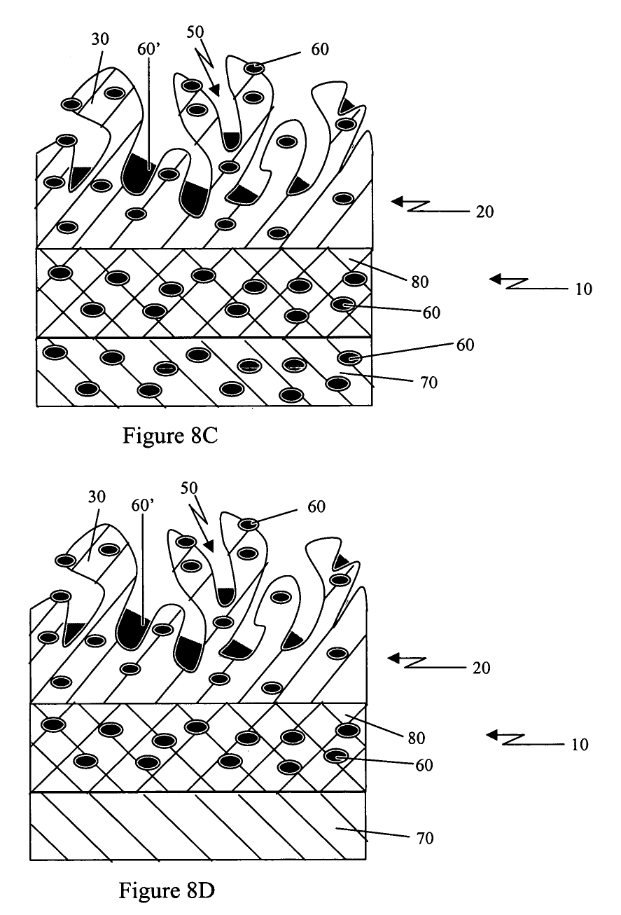

Polymeric material 20 as shown in FIGS. 1-5 may be disposed on or about at least a portion of a surface layer 70 of device 10. Examples of portions of such resulting devices 10 are shown in FIGS. 6-8. As shown in FIGS. 6A-6C and 8A, polymeric material 20 may be disposed on surface layer 70. Alternatively, as illustrated in FIGS. 7A-7C and 8B-8D, an intermediate layer 80 may be disposed between polymeric material 20 and surface layer 70. It will be understood that two, three, four, five, or more intermediate layers 80 may be disposed between polymeric material 20 and surface layer 70. Intermediate layer may be formed of any material. Preferably, intermediate layer 80 is formed of biocompatible material. Intermediate layer 80 may comprise one or more polymers that may be the same or different from those of polymeric material 20. One or more intermediate layer 80 may comprise a porous or non-porous polymeric material. Therapeutic agent 60 placed in an intermediate porous layer 20 may be expected to be released into tissue more rapidly than if placed in a non-porous intermediate layer, as therapeutic agent 60 from an underlying porous layer should permeate through a porous polymer more rapidly than through a non-porous polymer. If an intermediate layer 80 is porous, therapeutic agent 60 may be disposed in pores (not shown) of the intermediate layer 80 and/or may be disposed in or on the polymeric matrix of the intermediate layer 80. Accordingly, the release profile of therapeutic agent 60 may be more finely controlled by selecting placement in pores 50, matrix 30 of porous polymeric material 20, and matrix or pores of underlying porous polymeric material. Therapeutic agent 60 may be disposed in or on surface layer 70 and/or intermediate layer 80, as shown in FIGS. 8A-8D.

As shown in FIG. 6C, polymeric material 20 comprising polymeric matrix 30, therapeutic agent 60 in or on matrix 30, pores 50, and additional therapeutic agent 60′ may be disposed on surface layer 70 of device 10. Such a configuration may be desirable in many situations. For example, if therapeutic agent 60 or additional therapeutic agent 60′ is incompatible with surface layer 70, polymeric material 20 may serve as a buffer between surface layer 70 and therapeutic agent 60, 60′. If it is difficult to load sufficient quantities of therapeutic agent 60, 60′ on or in surface layer 70 or if it is difficult to control the release profile of therapeutic agent 60, 60′ from surface layer 70, polymeric material 20 may serve as a means to load and control release of sufficient quantities of therapeutic agent 60, 60′. If loading therapeutic agent 60, 60′ in or on surface layer 70 would impair the integrity of device 10, polymeric material 20 may serve as a means for maintaining the structural or functional integrity of surface layer 70 while still providing for release of therapeutic agent 60, 60′.

As shown in FIG. 7C, polymeric material 20 comprising polymeric matrix 30, therapeutic agent 60 in or on matrix 30, pores 50, and additional therapeutic agent 60′ may be disposed on intermediate layer 80, which is disposed on surface layer 70 of device 10. The presence of intermediate layer(s) 80, may be desirable in many situations. For example, intermediate layer(s) 80 may serve as a buffer between potentially incompatible therapeutic agent 60, 60′ and surface layer 70 or potentially incompatible polymeric material 20 and surface layer 20. Intermediate layer(s) 80 may serve to enhance the structural integrity of device 10. Further, as shown in FIGS. 8C and 8D, intermediate layer(s) 80 may serve as a means for loading and eluting therapeutic agent 60. The ability of intermediate layer(s) 80 to form a protective buffer, enhance integrity, or control release of therapeutic agent 60 will depend on the material from which intermediate layer(s) are formed, as well as the thickness and number of intermediate layers 80.

As shown in FIGS. 8A-8C, surface layer 70 of device may serve as a means for loading therapeutic agent 60. Release of therapeutic agent from surface layer 70 to tissue into which device 10 is implanted will likely occur more slowly than release from intermediate layer(s) 80 or polymeric material 20. Thus, the release profile of therapeutic agent 60, 60′ may be controlled by the amount of therapeutic agent 60, 60′ in or on surface layer 70, intermediate layer(s) 80, polymeric matrix 30, and pores 50.

Polymeric Material

Polymeric material 20 may be formed of any material capable of releasing therapeutic agent 60, 60′ into tissue when placed in contact with the tissue. Preferably, polymeric material is acceptable for at least temporary use within a human body. Polymeric material is also preferably compatible with therapeutic agent 60, 60′.

Examples of commonly used materials that may be used to form polymeric material 20 include organic polymers such as silicones, polyamines, polystyrene, polyurethane, acrylates, polysilanes, polysulfone, methoxysilanes, and the like. Other polymers that may be utilized include polyolefins, polyisobutylene and ethylene-alphaolefin copolymers; acrylic polymers and copolymers, ethylene-covinylacetate, polybutylmethacrylate; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; polycarbonates; polyoxymethylenes; polyimides; polyethers; epoxy resins; polyurethanes; rayon; rayon-triacetate; cellulose; cellulose acetate, cellulose butyrate; cellulose acetate butyrate; cellophane; cellulose nitrate; cellulose propionate; cellulose ethers; carboxymethyl cellulose; polyphenyleneoxide; and polytetrafluoroethylene (PTFE).

Polymeric material 20 according to various embodiments of the invention may comprise a biodegradable polymeric material, such as synthetic or natural bioabsorbable polymers. Synthetic bioabsorbable polymeric materials that can be used to form the coating layers include poly (L-lactic acid), polycaprolactone, poly(lactide-co-glycolide), poly(ethylene-vinyl acetate), poly(hydroxybutyrate-covalerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) such as PEO/PLA, polyalkylene oxalates, and polyphosphazenes. According to another exemplary embodiment, the polymeric materials can be natural bioabsorbable polymers such as, but not limited to, fibrin, fibrinogen, cellulose, starch, collagen, and hyaluronic acid.

Polymeric material 20 may be designed to control the rate at which therapeutic agent 60, 60′ is released, leached, or diffuses from the polymeric material. As used herein, “release”, “leach”, “diffuse”, “elute” and the like are used interchangeably when referring to a therapeutic agent 60. 60′ with respect to polymeric material 20, intermediate layer 80, or surface layer 70 of device 10. Any known or developed technology may be used to control the release rate. For example, a coating layer may be designed according to the teachings of WO/04026361, entitled “Controllable Drug Releasing Gradient Coating for Medical Devices.”

In an embodiment polymeric material 20 is formed from a non-biodegradable polymeric material, such as silicone or polyurethane.

Polymeric material 20 may be in the form of a tube, jacket, sheath, sleeve, cover, coating, or the like. Polymeric material 20 may be extruded, molded, coated on surface layer 70 or intermediate layer 80, grafted onto surface layer 70 or intermediate layer 80, embedded within surface layer 70 or intermediate layer 80, adsorbed to surface layer 70 or intermediate layer 80, etc. Polymers of polymeric material 20 may be porous, or may be made porous. Porous materials known in the art include those disclosed in U.S. Pat. No. 5,609,629 (Fearnot et al.) and U.S. Pat. No. 5,591,227 (Dinh et al.). Typically polymers are non-porous. However, non-porous polymers may be made porous through known or developed techniques, such as extruding with CO₂, by foaming the polymeric material prior to extrusion or coating, or introducing and then removing a porogen 40. Non-limiting examples of porogens 40 include salts, such as sodium bicarbonate, gelatin beads, sugar crystals, polymeric microparticles, and the like. One or more porogen 40 may be incorporated into a polymer prior to curing or setting. The polymer may then be cured or set, and the porogen 40 may be extracted with an appropriate solvent. Pores 50 generated by such techniques or processes typically range in size from between about 0.01 μm to about 100 μm. The size and degree of porosity of polymeric material 20 may be controlled by the size and concentration of porogen 40 used, the extent of mixing with gas or foaming, etc. Accordingly, the release profile of therapeutic agent 60, 60′ from polymeric material 20 may be controlled by varying the conditions under which pores 50 are generated, as pore size and degree of porosity are related to release rate. Larger pore 50 size, e.g., between about 1 μm and about 100 μm or between about 10 μm to 50 μm may be preferred when more rapid release of therapeutic agent 60 from polymeric material is desired.

Depending upon the type of materials used to form polymeric material 20, polymeric material 20 can be applied to the surface layer 70 or intermediate layer 80 through any coating processes known or developed in the art. One method includes directly bonding polymeric material 20 to surface layer 70 or underlying intermediate layer 80. By directly attaching a polymeric material 20 to surface layer 70 or intermediate layer 80, covalent chemical bonding techniques may be utilized. Surfaces of surface layer 70 or intermediate layer 80 may possess chemical functional groups, such as carbonyl groups, primary amines, hydroxyl groups, or silane groups which will form strong, chemical bonds with similar groups on polymeric material 20 utilized. In the absence of such chemical forming functional group, known techniques may be utilized to activate a material's surface before coupling the biological compound. Surface activation is a process of generating, or producing, reactive chemical functional groups using chemical or physical techniques such as, but not limited to, ionization, heating, photochemical activation, oxidizing acids, sintering, physical vapor deposition, chemical vapor deposition, and etching with strong organic solvents. Alternatively, polymeric material 20 may be indirectly bound to surface layer 70 or intermediate layer 80 through intermolecular attractions such as ionic or Van der Waals forces. Of course, if polymeric material 20 is in the form of a jacket, sheath, sleeve, cover, or the like, the chemical interaction between polymeric material 20 and surface layer 70 or intermediate layer 80 may be minimal.

Therapeutic agent 60, 60′ may be incorporated into polymeric material 20 in a variety of ways. For example, therapeutic agent 60, 60′ can be covalently grafted to a polymer of the polymeric material 20, either alone or with a surface graft polymer. Alternatively, therapeutic agent 60, 60′ may be coated onto the surface of the polymer either alone or intermixed with an overcoating polymer. Therapeutic agent 60, 60′ may be physically blended with a polymer of a polymeric material 20 as in a solid-solid solution. Therapeutic agent 60, 60′ may be impregnated into a polymer by swelling the polymer in a solution of the appropriate solvent. Any means of incorporating therapeutic agent 60, 60′ into or on a polymeric material 20 may be used, provided that therapeutic agent 60, 60′ may be released, leached or diffuse from polymeric material 20 on contact with bodily fluid or tissue.

A polymer of a polymeric material 20 and a therapeutic agent 60, 60′ may be intimately mixed either by blending or using a solvent in which they are both soluble. This mixture can then be formed into the desired shape or coated onto an underlying structure of the medical device. One exemplary method includes adding one or more therapeutic agent 60, 60′ to a solvated polymer to form a therapeutic agent 60, 60′/polymer solution. The therapeutic agent 60, 60′/polymer solution can then be applied directly to the surface layer 70 or intermediate layer 80; for example, by either spraying or dip coating device 10. As the solvent dries or evaporates, the therapeutic agent 60, 60′/polymer coating is deposited on device 10. Furthermore, multiple applications can be used to ensure that the coating is generally uniform and a sufficient amount of therapeutic agent 60, 60′ has been applied to device 10.

Alternatively, an overcoating polymer, which may or may not be the same polymer that forms the primary polymer of surface layer 70 (it will be understood that in some embodiments the external surface layer 12 of device 10 is formed of a polymeric material and in other embodiments the external surface layer 12 of device 10 is from non-polymeric material, such as metallic material) or intermediate layer 80, and therapeutic agent 60, 60′ are intimately mixed, either by blending or using a solvent in which they are both soluble, and coated onto surface layer 70 or intermediate layer 80. Any overcoating polymer may be used, as long as the polymer is able to bond (either chemically or physically) to the polymer of an underlying layer of device 10.

In addition, a polymer of a polymeric material 20 may be swelled with an appropriate solvent, allowing a therapeutic agent 60, 60′ to impregnate the polymer.

Therapeutic agent 60, 60′ may also be covalently grafted onto a polymer of a polymeric material 20. This can be done with or without a surface graft polymer. Surface grafting can be initiated by corona discharge, UV irradiation, and ionizing radiation. Alternatively, the ceric ion method, previously disclosed in U.S. Pat. No. 5,229,172 (Cahalan et al.), may be used to initiate surface grafting.

Additional therapeutic agent 60′ may be added to pores 50 by any known or future developed technique or procedure. For example, additional therapeutic agent 60′ may be added to pores 50 using a technique or process as described above. In an embodiment, additional therapeutic agent 60′ is disposed in pores 50 by contacting pores with a mixture comprising a solvent and additional therapeutic agent 60′. The solvent may be removed, by e.g. evaporation, leaving additional therapeutic agent 60′ disposed in pores 50. The solvent may or may not be a solvent that allows penetration of additional therapeutic agent 60′ into polymeric matrix 30.

Therapeutic Agent

Any therapeutic agent 60, 60′ may be disposed in or on polymeric matrix 30, pores 50, surface layer 70, or intermediate layer 80. Therapeutic agent 60 disposed in or on surface layer 70 may be the same or different than therapeutic agent 60 disposed in or on intermediate layer, which may be the same or different than therapeutic agent 60 disposed in or on polymeric matrix 30, which may be the same or different than additional therapeutic agent 60′. As used herein, “therapeutic agent 60” and “additional therapeutic agent 60′” are used interchangeably.

Because it may be desirable to treat or prevent infections and/or inflammation associated with implantation of a medical device 10, it may be desirable to dispose one or more anti-infective agent and/or one or more anti-inflammatory agent in, on, or about at least a portion of an external surface of device 10. In addition, in some circumstances it may be desirable to deliver a local anesthetic or antiproliferative agent. Additional agents that may be desirable disposed in or on polymeric matrix 30, pores 50, surface layer 70, or intermediate layer 80 will be readily evident to one of skill in the art. A brief summary of some non-limiting classes of therapeutic agents that may be used follows.

1. Anti-Infective Agents

Any anti-infective agent may be used in accordance with various embodiments of the invention. As used herein, “anti-infective agent” means an agent that kills or inhibits the growth of an infective organism, such as a microbe or a population of microbes. Anti-infective agents include antibiotics and antiseptics.

A. Antibiotic

Any antibiotic suitable for use in a human may be used in accordance with various embodiments of the invention. As used herein, “antibiotic” means an antibacterial agent. The antibacterial agent may have bateriostatic and/or bacteriocidal activities. Nonlimiting examples of classes of antibiotics that may be used include tetracyclines (e.g. minocycline), rifamycins (e.g. rifampin), macrolides (e.g. erythromycin), penicillins (e.g. nafcillin), cephalosporins (e.g. cefazolin), other beta-lactam antibiotics (e.g. imipenem, aztreonam), aminoglycosides (e.g. gentamicin), chloramphenicol, sufonamides (e.g. sulfamethoxazole), glycopeptides (e.g. vancomycin), quinolones (e.g. ciprofloxacin), fusidic acid, trimethoprim, metronidazole, clindamycin, mupirocin, polyenes (e.g. amphotericin B), azoles (e.g. fluconazole) and beta-lactam inhibitors (e.g. sulbactam). Nonlimiting examples of specific antibiotics that may be used include minocycline, rifampin, erythromycin, nafcillin, cefazolin, imipenem, aztreonam, gentamicin, sulfamethoxazole, vancomycin, ciprofloxacin, trimethoprim, metronidazole, clindamycin, teicoplanin, mupirocin, azithromycin, clarithromycin, ofloxacin, lomefloxacin, norfloxacin, nalidixic acid, sparfloxacin, pefloxacin, amifloxacin, enoxacin, fleroxacin, temafloxacin, tosufloxacin, clinafloxacin, sulbactam, clavulanic acid, amphotericin B, fluconazole, itraconazole, ketoconazole, and nystatin. Other examples of antibiotics, such as those listed in Sakamoto et al., U.S. Pat. No. 4,642,104, which is herein incorporated by reference in its entirety, may also be used. One of ordinary skill in the art will recognize other antibiotics that may be used.

In general, it is desirable that the selected antibiotic(s) kill or inhibit the growth of one or more bacteria that are associated with infection following surgical implantation of a medical device. Such bacteria are recognized by those of ordinary skill in the art and include Stapholcoccus aureus, Staphlococcus epidermis, and Escherichia coli. Preferably, the antibiotic(s) selected are effective against strains of bacteria that are resistant to one or more antibiotic.

To enhance the likelihood that bacteria will be killed or inhibited, it may be desirable to combine two or more antibiotics. It may also be desirable to combine one or more antibiotic with one or more antiseptic. It will be recognized by one of ordinary skill in the art that antimicrobial agents having different mechanisms of action and/or different spectrums of action may be most effective in achieving such an effect. In an embodiment, a combination of rifampin and micocycline is used. In an embodiment, a combination of rifampin and clindamycin is used.

B. Antiseptic

Any antiseptic suitable for use in a human may be used in accordance with various embodiments of the invention. As used herein, “antiseptic” means an agent capable of killing or inhibiting the growth of one or more of bacteria, fungi, or viruses. Antiseptic includes disinfectants. Nonlimiting examples of antiseptics include hexachlorophene, cationic bisiguanides (i.e. chlorhexidine, cyclohexidine) iodine and iodophores (i.e. povidone-iodine), para-chloro-meta-xylenol, triclosan, furan medical preparations (i.e. nitrofurantoin, nitrofurazone), methenamine, aldehydes (glutaraldehyde, formaldehyde), silver-containing compounds (silver sulfadiazene, silver metal, silver ion, silver nitrate, silver acetate, silver protein, silver lactate, silver picrate, silver sulfate), and alcohols. One of ordinary skill in the art will recognize other antiseptics that may be employed in accordance with this disclosure.

It is desirable that the antiseptic(s) selected kill or inhibit the growth of one or more microbe that are associated with infection following surgical implantation of a medical device. Such microbes are recognized by those of ordinary skill in the art and include Stapholcoccus aureus, Staphlococcus epidermis, Escherichia coli, Pseudomonus auruginosa, and Candidia.

To enhance the likelihood that microbes will be killed or inhibited, it may be desirable to combine two or more antiseptics. It may also be desirable to combine one or more antiseptics with one or more antibiotics. It will be recognized by one of ordinary skill in the art that antimicrobial agents having different mechanisms of action and/or different spectrums of action may be most effective in achieving such an effect. In a particular embodiment, a combination of chlorohexidine and silver sulfadiazine is used.

C. Antiviral

Any antiviral agent suitable for use in a human may be used in accordance with various embodiments of the invention. Nonlimiting examples of antiviral agents include acyclovir and acyclovir prodrugs, famcyclovir, zidovudine, didanosine, stavudine, lamivudine, zalcitabine, saquinavir, indinavir, ritonavir, n-docosanol, tromantadine and idoxuridine. One of ordinary skill in the art will recognize other antiviral agent that may be employed in accordance with this disclosure.

To enhance the likelihood that viruses will be killed or inhibited, it may be desirable to combine two or more antiviral agents. It may also be desirable to combine one or more antiseptics with one or more antiviral agent.

D. Anti-Fungal

Any anti-fungal agent suitable for use in a human may be used in accordance with various embodiments of the invention. Nonlimiting examples of anti-fungal agents include amorolfine, isoconazole, clotrimazole, econazole, miconazole, nystatin, terbinafine, bifonazole, amphotericin, griseofulvin, ketoconazole, fluconazole and flucytosine, salicylic acid, fezatione, ticlatone, tolnaftate, triacetin, zinc, pyrithione and sodium pyrithione. One of ordinary skill in the art will recognize other anti-fungal agents that may be employed in accordance with this disclosure.

To enhance the likelihood that viruses will be killed or inhibited, it may be desirable to combine two or more anti-fungal agents. It may also be desirable to combine one or more antiseptics with one or more anti-fungal agent.

2. Anti-Inflammatory Agents

Any anti-inflammatory agent suitable for use in a human may be used in accordance with various embodiments of the invention. Non-limiting examples of anti-inflammatory agents include steroids, such as cortisone, hydrocortisone, prednisone, dexamethasone, methyl-prednisilone, an derivatives thereof; and non-steroidal anti-inflammatory agents (NSAIDs). Non-limiting examples of NSAIDS include ibuprofen, flurbiprofen, ketoprofen, aclofenac, diclofenac, aloxiprin, aproxen, aspirin, diflunisal, fenoprofen, indomethacin, mefenamic acid, naproxen, phenylbutazone, piroxicam, salicylamide, salicylic acid, sulindac, desoxysulindac, tenoxicam, tramadol, ketoralac, flufenisal, salsalate, triethanolamine salicylate, aminopyrine, antipyrine, oxyphenbutazone, apazone, cintazone, flufenamic acid, clonixerl, clonixin, meclofenamic acid, flunixin, coichicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, and triflumidate.

3. Local Anesthetics

Any local anesthetic agent suitable for use in a human may be used in accordance with various embodiments of the invention. Non-limiting examples of local anesthetics agents include lidocaine, prilocalne, mepivicaine, benzocaine, bupivicaine, amethocaine, lignocaine, cocaine, cinchocaine, dibucaine, etidocaine, procaine, veratridine (selective c-fiber blocker) and articaine.

4. Anti-Proliferative Agents

Any local anti-proliferative agent suitable for use in a human may be used in accordance with various embodiments of the invention. As used herein, “anti-proliferative agents” includes anti-migration agents. In an embodiment, an anti-proliferative agent is an agent capable of preventing restenosis.

Examples of anti-proliferative agents include QP-2 (taxol), paclitaxel, rapamycin, tacrolimus, everolimus, actinomycin, methotrexate, angiopeptin, vincristine, mitocycin, statins, C-MYC antisense, sirolimus, restenASE, 2-chloro-deoxyadenosine, PCNA (proliferating cell nuclear antigent) ribozyme, batimastat, prolyl hydroxylase inhibitors, halofuginone, C-proteinase inhibitors, probucol, and combinations and/or derivates thereof. In an embodinent, one or more anti-proliferative agent with one or more anti-inflammatory agent.

5. Other Pharmacological Agents

Non-limiting examples of other pharmacological agents that may be used include: beta-radiation emitting isotopes, beclomethasone, fluorometholone, tranilast, ketoprofen, curcumin, cyclosporin A, deoxyspergualin, FK506, sulindac, myriocin, 2-aminochromone (U-86983), colchicines, pentosan, antisense oligonucleotides, mycophenolic acid, etoposide, actinomycin D, camptothecin, carmustine, methotrexate, adriamycin, mitomycin, cis-platinum, mitosis inhibitors, vinca alkaloids, tissue growth factor inhibitors, platinum compounds, cytotoxic inhibitors, alkylating agents, antimetabolite agents, tacrolimus, azathioprine, recombinant or monoclonal antibodies to interleukins, T-cells, B-cells, and receptors, bisantrene, retinoic acid, tamoxifen, compounds containing silver, doxorubicin, azacytidine, homoharringtonine, selenium compounds, superoxide-dismutase, interferons, heparin; Antineoplastic/antiangiogenic agents, such as antimetabolite agents, alkylating agents, cytotoxic antibiotics, vinca alkaloids, mitosis inhibitors, platinum compounds, tissue growth factor inhibitors, cisplatin and etoposide; Immunosuppressant agents, such as cyclosporine A, mycophenolic acid, tacrolimus, rapamycin, rapamycin analogue (ABT-578) produced by Abbott Laboratories, azathioprine, recombinant or monoclonal antibodies to interleukins, T-cells, B-cells and/or their receptors; Anticoagulents, such as heparin and chondroiten sulfate; Platelet inhibitors such as ticlopidine; Vasodilators such as cyclandelate, isoxsuprine, papaverine, dipyrimadole, isosorbide dinitrate, phentolamine, nicotinyl alcohol, co-dergocrine, nicotinic acid, glycerl trinitrate, pentaerythritol tetranitrate and xanthinol; Thrombolytic agents, such as stretokinase, urokinase and tissue plasminogin activators; and Analgesics and antipyretics, such as the opioid analgesics such as buprenorphine, dextromoramide, dextropropoxyphene, fentanyl, alfentanil, sufentanil, hydromorphone, methadone, morphine, oxycodone, papaveretum, pentazocine, pethidine, phenopefidine, codeine dihydrocodeine; acetylsalicylic acid (aspirin), paracetamol, and phenazone.

Surface Layer

Surface layer 70 of device 10 may be made of any material of which a surface of a medical device is made. Preferably, surface layer 70 is formed of material acceptable for at least temporary use within a human body. In an embodiment, surface layer 70 is formed of a polymer or combination of polymers, such as described above for polymeric material 20. In an embodiment, surface layer 70 is formed of a metallic material such as, but not limited to, stainless steel, MP35N alloy, superelastic Nitinol nickel-titanium, titanium alloys, and other alloys such as a wrought Cobalt-Chromium-Nickel-Molybdenum-iron alloy. When formed of a metallic material, surface layer 70 may be treated by, e.g., ionization, heating, photochemical activation, oxidizing acids, sintering, physical vapor deposition, chemical vapor deposition and/or etching with strong organic solvents, as discussed above, to facilitate disposing therapeutic agent 60, intermediate layer 80, or polymeric material 20 on surface layer 70.

Methods

Various embodiments of the invention provide methods for making medical devices 10 comprising a polymeric material 20, which comprises a polymeric matrix 30, therapeutic agent 60 disposed in or on the matrix 30, pores 50, and additional therapeutic agent 60′ disposed in the pores 50. In an embodiment, the polymeric material is the device 10, or a portion thereof. The devices 10 may be manufactured as generally described herein.

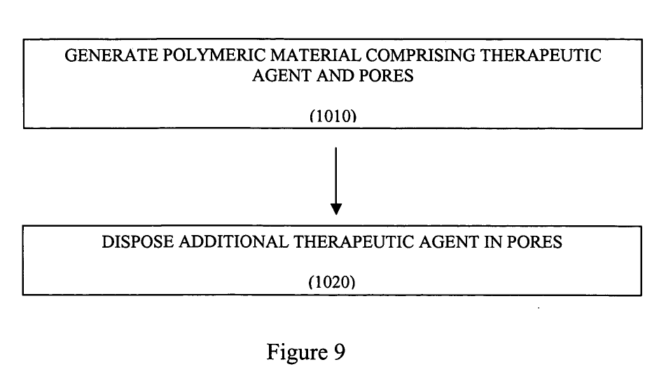

Referring to FIG. 9, an exemplary method is shown. The method comprises generating a polymeric material 20 comprising a polymeric matrix 30, therapeutic agent 60 disposed in or on the matrix 30, and pores 50 (1010). The method further comprises disposing additional therapeutic agent 60′ in the pores 50 (1020). In the method illustrated in FIG. 9, the surface layer 70 of device 10, or a portion thereof, is the polymeric material 20.

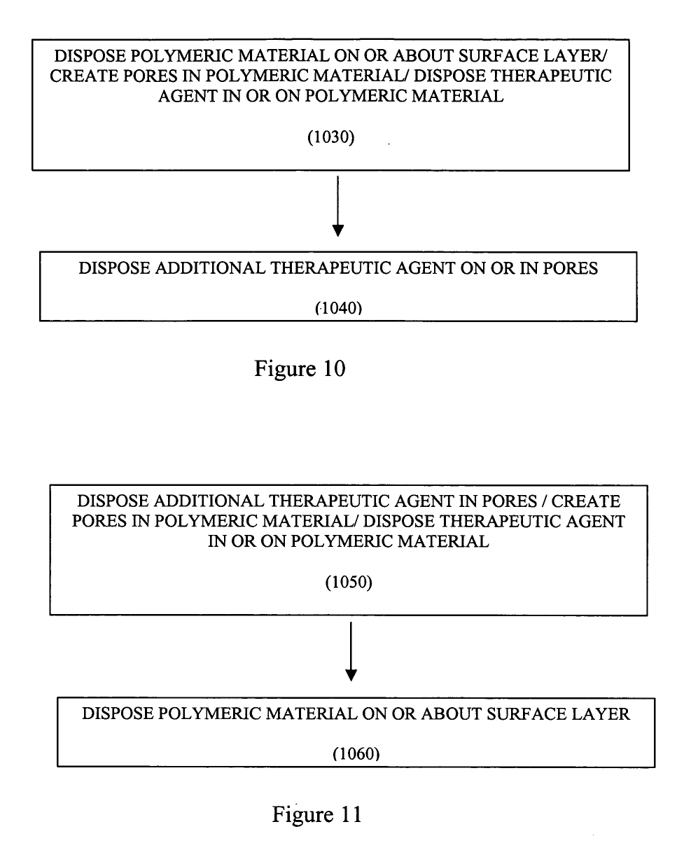

Referring to FIG. 10, another exemplary method is illustrated. The method comprises disposing polymeric material 20 on or about at least a portion of surface layer 70, creating pores 50 in polymeric material 20, and disposing therapeutic agent 60 in or on polymeric material 20 (1030). The processes described in step 1030 may be performed in any order. The method further comprises disposing additional therapeutic agent 60′ on or in the pores 50 (1040).

Referring to FIG. 11, another exemplary method is illustrated. The method comprises disposing additional therapeutic agent 60′ in pores 50, creating pores 50 in polymeric material 20, and disposing therapeutic agent 60 in or on polymeric material 20 (1050). The processes described in step 1050 may be performed in any order. The method further comprises disposing the polymeric material 20 resulting from step 1050 on or about at least a portion of surface layer 70 of device.

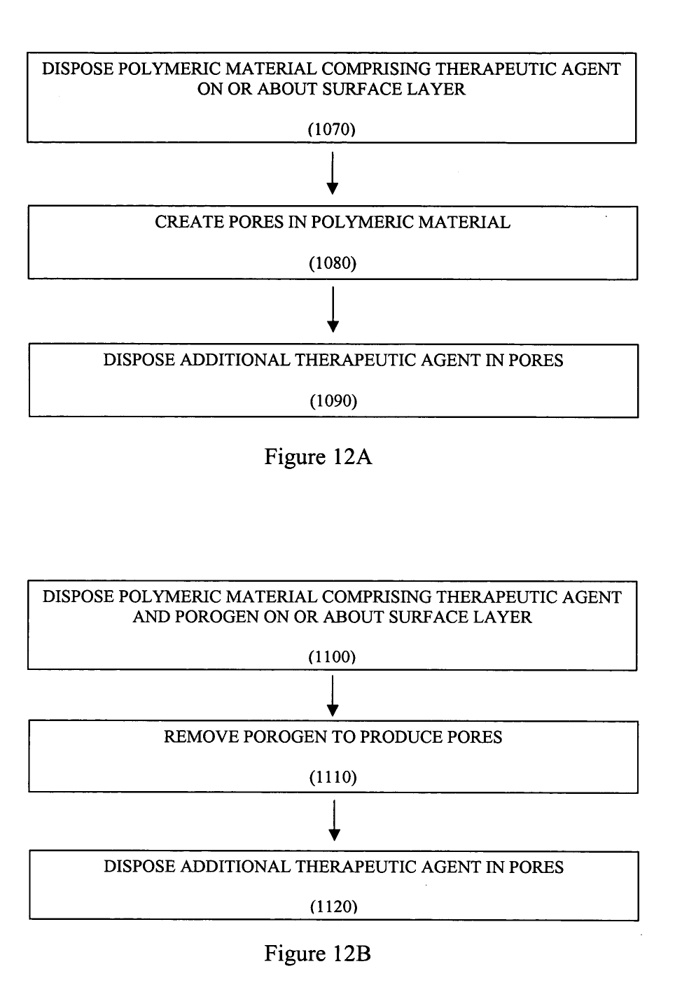

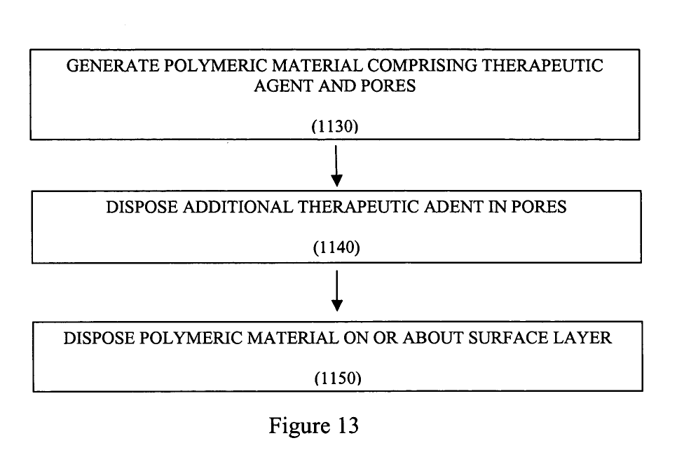

FIG. 12A illustrates another exemplary method. In step 1070, polymeric material 20 is disposed on or about at least a portion of surface layer 70. In step 1080, pores 50 are created in the polymeric material 20. In step 1090, additional therapeutic agent is disposed in the pores

FIG. 12B illustrates yet another exemplary method. The method comprises disposing polymeric material 20 comprising a therapeutic agent 60 and a porogen 40 on or about at least a portion of surface layer 70 (1100). The polymeric material 20 may be silicon RTV.

The porogen 40 may be sodium bicarbonate. The therapeutic agent 60, porogen 40, and polymeric matrix 30 material may be a solution or mixture in tetrahydrofuran (THF). The surface layer 70, or portion thereof, may be dipped into the solution or mixture. The resulting device may be dried and cured to produce a device 10 comprising a polymeric material 20 disposed on a surface layer 70, or portion thereof. The polymeric material 20, at this point, comprises a polymeric matrix 30, therapeutic agent 60 disposed therein, and porogen 40 (sodium bicarbonate). The method further comprises removing the porogen 40 from the polymeric material 20 to produce pores 50 (1110). This may be done by contacting the polymeric material 70 with an appropriate solvent. For example deionized water may be used to extract sodium bicarbonate from silicone. The method further comprises disposing additional therapeutic agent 60′ from pores 50 (1120). This may be done by, e.g., contacting polymeric material 20 with a solution or mixture of additional therapeutic agent in a solvent and drying or evaporating the solvent, leaving additional therapeutic agent 60′ in pores 50. By way of example, dexamethasone in an acetone solvent may be contacted with the polymeric material 20 from which the sodium bicarbonate was extracted.

FIG. 12C illustrates still another exemplary method. In step 1130, polymeric material 20 comprising therapeutic agent 60 and pores 50 is generated. In step 1140, additional therapeutic agent 60′ is disposed in pores 50. In step 1150, the resulting polymeric material is disposed on or about at least a portion of surface layer 70.

Of course, many other general and specific methods are contemplated and will be readily evident to one skilled in the art upon reading the present disclosure.

Various embodiments of the invention are disclosed. One skilled in the art will appreciate that the present invention can be practiced with embodiments other than those disclosed. The disclosed embodiments are presented for purposes of illustration and not limitation.

All printed publications, such as patents, technical papers, and brochures, and patent applications cited herein are hereby incorporated by reference herein, each in its respective entirety. As those of ordinary skill in the art will readily appreciate upon reading the description herein, at least some of the devices and methods disclosed in the patents and publications cited herein may be modified advantageously in accordance with the teachings of the present invention.

What is claimed is:

1. A method for manufacturing a medical device, comprising:
generating a polymeric material comprising therapeutic agent and pores; and
disposing additional therapeutic agent in the pores,
wherein at least a portion of a structural surface layer of the device comprises the polymeric material comprising therapeutic agent and pores,
wherein the therapeutic agent and the additional therapeutic agent are the same or different.

2. The method of claim 1, wherein the generating the polymeric material comprises:
blending an uncured or unset polymer with the therapeutic agent and a porogen to produce a mixture;
curing or setting the mixture; and
removing the porogen to generate the polymeric material comprising therapeutic agent and pores.

3. The method of claim 1, wherein the generating the polymeric material comprises:
blending an uncured or unset polymer with a porogen to produce a mixture;
curing or setting the mixture to produce a material comprising pores;
impregnating the material comprising pores with the therapeutic agent to generate the polymeric material comprising therapeutic agent and pores.

4. The method of claim 3, wherein the impregnating the material comprising pores with therapeutic agent comprises:
swelling the material comprising pores; and
introducing therapeutic agent into the swelled material via a solvent vehicle.

5. The method of claim 1, wherein the generating the polymeric material comprises:
mixing an uncured or unset polymer with a gas;
curing or setting the polymer and removing the gas to generate a material comprising pores;
impregnating the material comprising pores with the therapeutic agent to generate the polymeric material comprising therapeutic agent and pores.

6. The method of claim 5, wherein the impregnating the material comprising pores with therapeutic agent comprises:
swelling the material comprising pores; and
introducing therapeutic agent into the swelled material via a solvent vehicle.

7. The method of claim 5, wherein the mixing the uncured or unset polymer with a gas comprises foaming the uncured or unset polymer.

8. The method of claim 1, wherein the generating the polymeric material comprises:
mixing an uncured or unset polymer with a gas and the therapeutic agent;
curing or setting the polymer and removing the gas to generate the polymeric material comprising therapeutic agent and pores.

9. The method of claim 5, wherein the mixing the uncured or unset polymer with a gas comprises foaming the uncured or unset polymer.

10. The method of claim 1, wherein the disposing additional therapeutic agent in the pores comprises:
contacting the polymeric material comprising therapeutic agent and pores with a solution or mixture comprising the additional therapeutic agent and a solvent; and
removing the solvent to deposit additional therapeutic agent in the pores.

11. The method of claim 10, wherein the contacting the polymeric material with the solution or mixture swells the polymeric material.

12. The method of claim 11, wherein at least a portion of the additional therapeutic agent impregnates the polymeric material.

13. The method of claim 10, wherein contacting the polymeric material with the solution of mixture does not substantially swell the polymeric material.

14. The method of claim 1, wherein the generating a polymeric material comprising therapeutic agent and pores comprises:
generating a polymeric material comprising pores and a therapeutic agent selected from the group consisting of one or more anti-infective agent, one or more anti-inflammatory agent, one or more local anesthetic, one or more anti-proliferative agent, and a combination thereof.

15. The method of claim 14, wherein the generating a polymeric material comprising therapeutic agent and pores comprises:
generating a polymeric material comprising one or more anti-infective agent and one or more anti-inflammatory agent.

16. The method of claim 14, wherein the generating a polymeric material comprising therapeutic agent and pores comprises:
generating a polymeric material comprising one or more anti-inflammatory agent and one or more anti-proliferative agent.

17. The method of claim 14, wherein the generating a polymeric material comprising therapeutic agent and pores comprises:
generating a polymeric material comprising minocycline, rifampin, chlorhexidine, clindamycin, a silver-containing compound, or combinations thereof.

18. The method of claim 17, wherein the generating a polymeric material comprising therapeutic agent and pores comprises:
generating a polymeric material comprising minocycline and rifampin.

19. The method of claim 17, wherein the generating a polymeric material comprising therapeutic agent and pores comprises:
generating a polymeric material comprising chlorhexidine and silver sulfadiazine.

20. The method of claim 17, wherein the generating a polymeric material comprising therapeutic agent and pores comprises:
generating a polymeric material comprising clindamycin and rifampin.

21. The method of claim 1, wherein the disposing additional therapeutic agent in the pores comprises:
disposing in the pores an agent selected from the group consisting of one or more anti-infective agent, one or more anti-inflammatory agent, one or more local anesthetic, one or more anti-proliferative agent, and combinations thereof.

22. The method of claim 21, wherein the disposing additional therapeutic agent in the pores comprises:
disposing in the pores one or more anti-infective agent and one or more anti-inflammatory agent.

23. The method of claim 21, wherein the disposing additional therapeutic agent in the pores comprises:
disposing in the pores one or more anti-inflammatory agent and one or more anti-proliferative agent.

24. The method of claim 21, wherein the disposing additional therapeutic agent in the pores comprises:
disposing in the pores minocycline, rifampin, chlorhexidine, clindamycin, a silver-containing compound, or combinations thereof.

25. The method of claim 24, wherein the disposing additional therapeutic agent in the pores comprises:
disposing in the pores minocycline and rifampin.

26. The method of claim 24, wherein the disposing additional therapeutic agent in the pores comprises:
disposing in the pores chlorhexidine and silver sulfadiazine.

27. The method of claim 24, wherein the disposing additional therapeutic agent in the pores comprises:
disposing in the pores clindamycin and rifampin.

28. The method of claim 1, wherein the generating a polymeric material comprising therapeutic agent and pores comprises:
generating a polymeric material comprising silicone.

29. The method of claim 28, wherein the silicon is silicone RTV.

30. The method of claim 1, wherein the generating a polymeric material comprising therapeutic agent and pores comprises:
generating a polymeric material comprising polyurethane.

31. A method for manufacturing a medical device, comprising:
disposing therapeutic agent into or on a polymeric material;
creating pores in the polymeric material, disposing the polymeric material on or about a surface layer of the device; and
disposing additional therapeutic agent in the pores.

32. The method of claim 31, wherein the disposing the polymeric material on or about a at least a portion of a surface layer of the device comprises disposing a polymeric material comprising therapeutic agent and pores and additional therapeutic agent disposed in the pores on or about the device.

33. The method of claim 31, wherein disposing the additional therapeutic agent in the pores comprises disposing additional therapeutic agent in the pores of a polymeric material disposed on or about at least a portion of a surface layer of the device, the polymeric material comprising therapeutic agent and the pores.

34. The method of claim 31, further comprising disposing one or more intermediate layer on at least a portion of the surface layer of the device and disposing the polymeric material one of the one or more intermediate layers.

35. The method of claim 31, wherein the surface layer comprises metallic material.

36. The method of claim 31, wherein the surface layer comprises a polymer.

37. The method of claim 31, wherein the disposing therapeutic agent into or on a polymeric material comprises:
blending an uncured or unset polymer with the therapeutic agent to produce a mixture; and
curing or setting the mixture.

38. The method of claim 31, wherein the disposing therapeutic agent into or on a polymeric material comprises:
impregnating the polymeric material with the therapeutic agent.

39. The method of claim 38, wherein the impregnating the polymeric material with the therapeutic agent comprises:
swelling the polymeric material; and
introducing therapeutic agent into the swelled polymeric material via a solvent vehicle.

40. The method of claim 31, wherein the creating pores in the polymeric material comprises:
blending an uncured or unset polymeric material with a porogen;
curing or setting the polymeric material; and
removing the porogen to create pores in the polymeric material.

41. The method of claim 31, wherein the creating pores in the polymeric material comprises:
mixing an uncured or unset polymeric material with a gas;
curing or setting the polymeric and removing the gas to generate a polymeric material comprising pores.

42. The method of claim 41, wherein the mixing an uncured or unset polymeric material with a gas comprises foaming the uncured or unset polymeric material.

43. The method of claim 31, wherein the disposing additional therapeutic agent in the pores comprises:
contacting polymeric material comprising pores with a solution or mixture comprising the additional therapeutic agent and a solvent; and
removing the solvent to deposit additional therapeutic agent in the pores.

44. The method of claim 43, wherein the contacting the polymeric material with the solution or mixture swells the polymeric material.

45. The method of claim 44, wherein at least a portion of the additional therapeutic agent impregnates the polymeric material.

46. The method of claim 43, wherein contacting the polymeric material with the solution of mixture does not substantially swell the polymeric material.

47. The method of claim 31, wherein the disposing therapeutic agent into or on a polymeric material comprises:
disposing into or on the polymeric material a therapeutic agent selected from the group consisting of one or more anti-infective agent, one or more anti-inflammatory agent, one or more local anesthetic, one or more anti-proliferative agent, and combinations thereof.

48. The method of claim 47, wherein the disposing therapeutic agent into or on a polymeric material comprises:
disposing into or on the polymeric material one or more anti-infective agent and one or more anti-inflammatory agent.

49. The method of claim 47, wherein the disposing therapeutic agent into or on a polymeric material comprises:
disposing into or on the polymeric material one or more anti-inflammatory agent and one or more anti-proliferative agent.

50. The method of claim 47, wherein the disposing therapeutic agent into or on a polymeric material comprises:
disposing into or on the polymeric material minocycline, rifampin, chlorhexidine, clindamycin, a silver-containing compound, or combinations thereof.

51. The method of claim 50, wherein the disposing therapeutic agent into or on a polymeric material comprises:
disposing into or on the polymeric material minocycline and rifampin.

52. The method of claim 50, wherein the disposing therapeutic agent into or on a polymeric material comprises:
disposing into or on the polymeric material chlorhexidine and silver sulfadiazine.

53. The method of claim 50, wherein the disposing therapeutic agent into or on a polymeric material comprises:
disposing into or on the polymeric material clindamycin and rifampin.

54. The method of claim 31, wherein the disposing additional therapeutic agent in the pores comprises:
disposing in the pores an agent selected from the group consisting of one or more anti-infective agent, one or more anti-inflammatory agent, one or more local anesthetic, one or more anti-proliferative agent, and a combination thereof.

55. The method of claim 54, wherein the disposing additional therapeutic agent in the pores comprises:
disposing in the pores one or more anti-infective agent and one or more anti-inflammatory agent.

56. The method of claim 54, wherein the disposing additional therapeutic agent in the pores comprises:
disposing in the pores one or more anti-inflammatory agent and one or more anti-proliferative agent.

57. The method of claim 54, wherein the disposing additional therapeutic agent in the pores comprises:
disposing in the pores minocycline, rifampin, chlorhexidine, clindamycin, a silver-containing compound, or combinations thereof.

58. The method of claim 57, wherein the disposing additional therapeutic agent in the pores comprises:
disposing in the pores minocycline and rifampin.

59. The method of claim 57, wherein the disposing additional therapeutic agent in the pores comprises:
disposing in the pores chlorhexidine and silver sulfadiazine.

60. The method of claim 57, wherein the disposing additional therapeutic agent in the pores comprises:
disposing in the pores clindamycin and rifampin.

61. The method of claim 31, wherein the polymeric material comprises silicone.

62. The method of claim 61, wherein the silicon is silicone RTV.

63. The method of claim 31, wherein the polymeric material comprises polyurethane.
